(19) United States
(12) Patent Application Publication
(10) Pub. No.: US 2021/0030513 A1
(43) Pub. Date: Feb. 4, 2021

(54) Surgical Instrument

(71) Applicant: Pajunk GmbH Medizintechnologie, Geisingen (DE)

(72) Inventors: Simone Pajunk-Schelling, Geisingen (DE); Martin Hauger, Donaueschingen (DE)

(73) Assignee: Pajunk GmbH Medizintechnologie, Geisingen (DE)

(21) Appl. No.: 16/943,748

(22) Filed: Jul. 30, 2020

(30) Foreign Application Priority Data
Jul. 31, 2019 (DE) 10 2019 120 671.5

(51) Int. Cl. A61B 90/00; A61B 17/32

(52) U.S. Cl. CPC A61B 90/39; A61B 8/481; A61B 2090/3925; A61B 17/320016

(57) ABSTRACT

A surgical instrument with a handle and a blade disposed thereon having a concavely developed cutting edge, wherein the blade comprises a plane, wherein the surgical instrument comprises ultrasound visibility-enhancing media.

### CROSS-REFERENCE TO RELATED APPLICATIONS

This application claims priority to German Patent Application No. 10 2019 120 671.5, filed Jul. 31, 2019, which is incorporated by reference in its entirety.

### BACKGROUND

The present application relates to a surgical instrument.

### SUMMARY

The median nerve of the hand that innervates muscles of the thumb and the metacarpus passes through a tunnel-like tube, the so-called carpal tunnel. At the level of the wrist stretches a broad ligament between the muscles of the thumb and little finger. This ligament, also referred to as retinaculum flexorum, forms the roof of the carpal tunnel that contains the median nerve and the flexor tendons of the fingers. In carpal tunnel syndrome this channel becomes too constricted and pressure is exerted onto the median nerve. This can lead to discomfort in the form, for example, of tingling or numbness of the hand.

In some cases surgical intervention is required for the treatment of carpal tunnel syndrome. This surgery involves widening the nerve channel by severing the retinaculum flexorum which stretches over the carpal bones and delimits the carpal tunnel on the palmar aspect. This intervention can be carried out as an open as well as also a minimally invasive surgical procedure. In a possible procedure for minimally invasive carpal roof release an incision of approximately two centimeters is placed in the flexor-side aspect of the wrist. A small probe with a small knife is introduced through the incision between the median nerve and the carpal roof into the carpal tunnel. When the probe is placed in the carpal tunnel, the carpal roof can be released from the inside. In an alternative surgical method a two to three cm long incision is placed at one end of the carpal tunnel and subsequently the visible portion of the transverse wrist ligament is transected under direct view. The remaining portion of the ligament can be transected either with scissors or a special knife without direct view, thus blind.

For minimally invasive interventions a surgical instrument with a handle and a blade having a concavely developed cutting edge disposed thereon is known, wherein the blade has a plane. Entailed in the minimally invasive intervention is the disadvantage that the blade must be advanced at a short distance from the nerves extending in the hand such that there is the risk of damaging the nerves.

The present disclosure therefore addresses the problem of providing a surgical instrument with which the safety of the minimally invasive intervention on the carpal tunnel of the hand can be increased.

The problem addressed by the present disclosure is resolved through a surgical instrument having the features and structures recited herein.

The surgical instrument according to the present disclosure with a handle and a blade disposed thereon having a concavely developed cutting edge, wherein the blade comprises a plane, is distinguished thereby that the surgical instrument comprises ultrasound visibility-enhancing means or media. By disposing media that increase the ultrasound visibility the surgical instrument becomes visible during the minimally invasive intervention such that the surgeon can detect at any point in time at which position the blade of the surgical instrument is located in the hand and whether or not there is a risk of damaging a nerve. Thereby timely correction of the advance movement of the surgical instrument is enabled as well as avoidance of nerve injury.

The media advantageously comprise ultrasound reflectors which enable an especially good increase of the ultrasound visibility.

According to an advantageous further development of the present disclosure, the ultrasound reflectors are developed as indentations. This permits a simple manufacture of the ultrasound visibility-enhancing media.

An especially preferred implementation of the present disclosure provides that the indentations comprise in each instance at least two, preferably precisely three, side faces oriented perpendicularly to one another. Such indentations permit an especially good ultrasound visibility. In particular, indentations with precisely three side faces oriented perpendicularly to one another enable good ultrasound visibility from different directions.

According to an advantageous further development of the present disclosure, the media are disposed nondetachably on the blade, for example they are adhered onto it or connected unitarily therewith. With such a disposition the relative orientation between the media and the blade is fixed.

A preferred embodiment of the present disclosure provides for the media to be, at least in sections, provided with a coating. The coating is implemented in particular such that it is transparent for ultrasound. Such a coating can improve cleanability of the surgical instrument since, for example, the indentations can be filled with the coating and the coating can have a smooth outer side.

The media are preferably, at least in sections, encapsulated with the blade. Thereby crevices or scratches in which contaminations can collect can be decreased or avoided.

The media preferably comprise a sheet-like base element which comprises a plane. Such a base element permits the intentional and specific orientation of the ultrasound visibility-enhancing media.

According to an advantageous further development of the present disclosure, the ultrasound reflectors are disposed on the base element. This enables simplicity of manufacture.

An especially preferred embodiment of the present disclosure provides that the plane of the sheet-like base element is substantially disposed perpendicularly to the plane of the blade. This enables especially good ultrasound visibility of the blade which during the operation is commonly disposed perpendicularly to the skin surface.

The ultrasound visibility is further improved if preferably a first of the side faces of one of the indentations forms an angle of approximately 35° with the plane of the base element.

A further improvement of the ultrasound visibility can be achieved if, advantageously, a second and a third of the side faces are disposed symmetrically with respect to a longitudinal axis of the base element.

The indentations advantageously comprise a triangular base area in the outer surface of the base element, wherein the apex of the triangle points toward a distal end of the surgical instrument. Such physical form can further improve the ultrasound visibility.

According to an especially preferred embodiment of the present disclosure, the surgical instrument is developed in the shape of a stylus with a distal and a proximal end, wherein the blade is disposed at the distal end, preferably frontally on the distal end. Such physical form is favorable for a good minimally invasive intervention.

The blade is preferably disposed on a blade holder.

According to an advantageous further development of the present disclosure, the media can also be disposed nondetachably on the blade holder instead of on the blade. They can, for example also be adhered thereon or be unitarily connected therewith. If the blade is fixed in the blade holder, the relative disposition between blade and media can also be ensured in this way.

The blade holder can be unitarily disposed on the handle. An especially advantageous embodiment of the present disclosure provides for the blade or the blade holder to be secured detachably on the handle, preferably by means of a threaded connection or a bayonet connection. A detachable securement enables an exchange and replacement. It is in particular enabled thereby for the blade or the blade holder including the blade to be implemented as a disposable product which can be disposed after single use, while the handle can be implemented as a reusable product.

The surgical instrument advantageously comprises a first section, which preferably is substantially formed by the handle, with a first longitudinal axis and a second section, which preferably is substantially formed by the blade holder, with a second longitudinal axis, wherein the second longitudinal axis is bent by an angle relative to the first longitudinal axis, which angle is in the range of 10° to 40°, preferably in the range of 20° to 30°, and especially preferably is 25°. Such an implementation is favorable for good minimally invasive intervention.

The angle by which the second longitudinal axis is bent with respect to the first longitudinal axis lies preferably in the plane of the blade whereby manipulation during the surgical procedure can be simplified.

### BRIEF DESCRIPTION OF THE DRAWINGS

An embodiment example of the present application will be described in detail in conjunction with the following Figures. Therein depict

FIG. 1 a perspective view of an embodiment example of a surgical instrument according to the present disclosure,

FIG. 2 an enlarged detail of the distal end of the surgical instrument according to FIG. 1,

FIG. 3 the distal end of the surgical instrument as depicted in FIG. 2 with the blade holder blanked out,

FIG. 4 a top view onto the distal end according to FIG. 3,

FIG. 5 a view from the front onto the distal end according to FIG. 3,

FIG. 6 a top view onto the surgical instrument according to FIG. 1,

FIG. 7 a section along line A-A in FIG. 6, and

FIG. 8 an enlarged detail from FIG. 3.

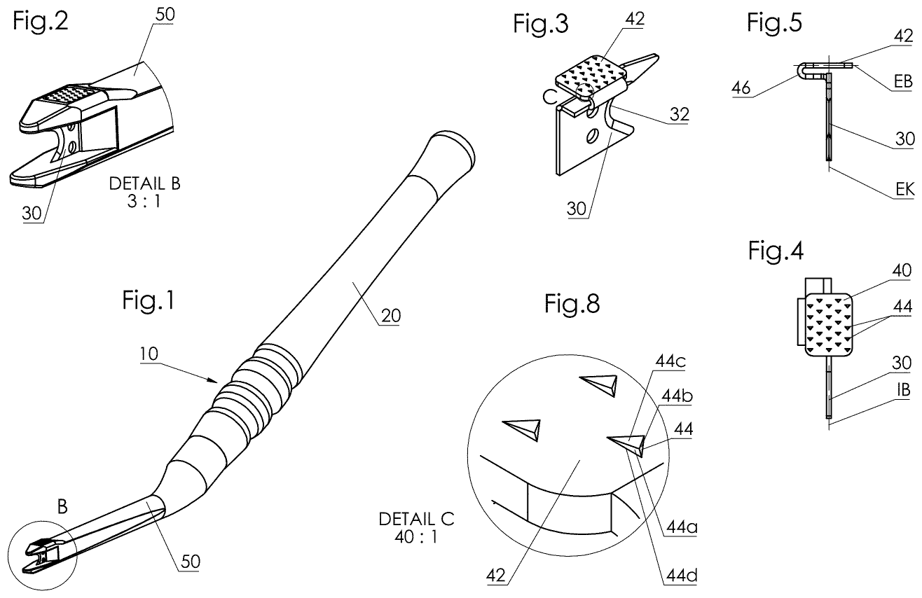

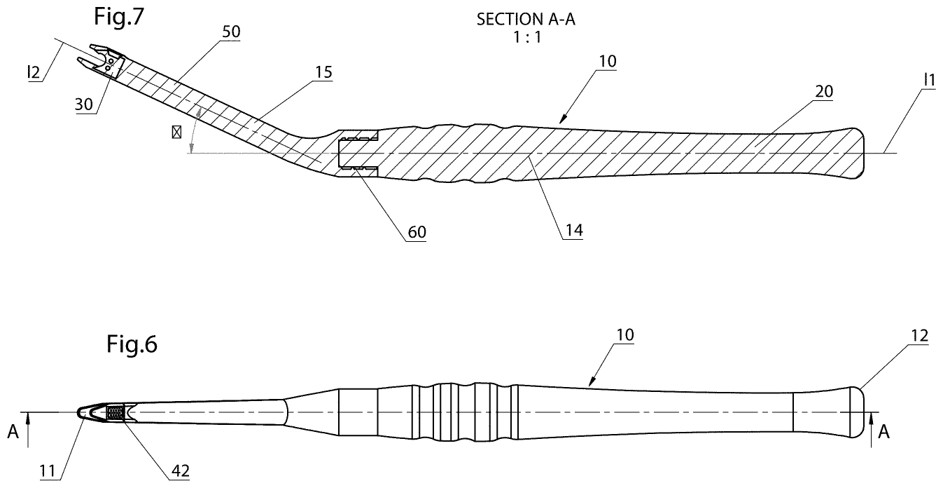

FIGS. 1 to 8 show different views of an embodiment example of a surgical instrument 10 according to the present application.

### DETAILED DESCRIPTION

The instrument 10 comprises a handle 20 and a blade 30 disposed thereon with a concavely developed cutting edge 32. The blade 30 can be developed knife-like and therein comprises a plane EK that extends parallel to a surface. In FIG. 7 the plane EK of blade 30 is the plane of the paper and is discernible in section in FIG. 5. The thickness of the knife-like blade should be sufficient to ensure adequate stability. The cutting edge 32 can be developed, for example, in the shape of a U or a V. The cutting edge 32 lies in particular in the plane EK of blade 30. The cutting edge 32 is developed for example, starting from one of the side edges of the knife-like blade 30, as a concave recess.

The blade 30 can be disposed directly on the handle 20. In the present embodiment example the blade 30 is secured in a blade holder 50 disposed on the handle 20. The blade holder 50 can herein be connected unitarily with the handle 20 or, as depicted in the Figures of the present embodiment example, be detachably disposed on the handle 20 via a detachable connection, for example via a threaded connection 60 or a bayonet connection. This permits the simple exchange of the blade 30 thereby that the blade 30 including the blade holder 50 can be exchanged.

The instrument 10 can comprise a first section 14, formed, for example, largely by the handle 20, with a first longitudinal axis l1, and a second section 15, formed largely by the blade holder 50, with a second longitudinal axis l2. The longitudinal axes l1 and l2 can be aligned. However, the second longitudinal axis l2 is, however, advantageously bent by an angle α with respect to the first longitudinal axis l1, wherein the angle α is in the range of 10° to 40°, preferably in the range of 20° to 30° and is, for example 25°. The angle α, by which the second longitudinal axis l2 is bent with respect to the first longitudinal axis l1, lies in particular in the plane EK of blade 30 (cf. FIG. 7).

The instrument 10 can be developed in the form of a stylus with a distal end 11 and a proximal end 12, wherein the blade 30 is disposed at the distal end 11, in particular is disposed frontally on the distal end 11. The blade 30 is herein oriented such that the longitudinal axis l2 of the first section, in particular of the handle 20, lies in the plane of the blade 30.

The surgical instrument 10 comprises ultrasound visibility-enhancing media 40. These can be disposed in the region of the distal end 11 or in the proximity thereto and are preferably disposed on the blade 30. The media 40 can be disposed nondetachably on the blade 30 or on the blade holder 50. To this end, the media 40 can be, for example, adhered to the blade 30 or be connected unitarily with the blade 30 or the blade holder 50. The media 40 can also be clipped on or snapped on. The media 40 can, at least in sections, be encapsulated with the blade 30 or the blade holder 50 in order to decrease or avoid cracks or crevices.

The media 40 comprise in particular ultrasound reflectors which can be implemented as indentations 44. The indentations 44 are in particular developed such that they reflect ultrasound, in particular independently of the direction from which the ultrasound impinges on the indentations 44. For this purpose, the indentations [44] can have, for example, precisely three side faces 44a, 44b, 44c oriented perpendicularly to one another (cf. in particular FIG. 8). This results in an indentation 44 such as if a corner of a cube had been pressed into a surface. Such indentations 44 reflect back an incident ultrasound beam, independently of the direction of incidence, parallel to the direction of incidence.

A first side face 44a of the indentation 44 can form an angle of approximately 35° with the plane EB of the base element 42. A second side face 44b and a third side face 44c can be disposed symmetrically to a longitudinal axis lB of the base element 42. For example, the indentation 44 can comprise a triangular base area 44d in the outer surface of base element 42, wherein the apex of the triangle points toward the distal end 11 of the surgical instrument [10] (cf. FIGS. 4 and 8).

The media 40 preferably comprise a sheet-like base element 42 which comprises a plane EB extending parallel to a surface of the sheet-like base element 42. The plane EB of base element [42] in FIG. 4 is the plane of the paper and is discernible in FIG. 5 in section. The plane EB of base element 42 extends in particular substantially perpendicularly to plane EK of blade 30. The indentations 44 are disposed in particular in the sheet-like base element 42. The sheet-like base element 42 should therefore be of an adequate thickness that is greater than the depth of the indentation 44. The base element 42 can be disposed directly on one of the side edges of the knife-like blade 30. The base element 42 can alternatively, as is especially discernible in FIG. 5, be disposed by means of a connection bracket 46 on one of the side edges of the knife-like blade 30. The length of base element 42 can herein be longer or shorter than the length of blade 30 or can correspond thereto.

The media 40 can be provided at least in sections or entirely with a coating which, in particular, fills the indentations 44 and forms a smooth outer side which is easily cleanable. The media 40 can be fabricated in particular of metal, while the coating can be fabricated, for example of an ultrasound-transparent material. The blade holder 50 and/or the handle 20 can be produced of synthetic material, for example utilizing an injection molding process, wherein, in particular, the blade holder 50 and the handle 20 can be developed as two parts or they can also be unitarily connected with one another.

### LIST OF REFERENCE SYMBOLS

10 Instrument
11 Distal end
12 Proximal end
14 First section
15 Second section
20 Handle
30 Blade
32 Cutting edge
40 Media that enhance ultrasound visibility
42 Base element
44 Indentation
44a First side surface
44b Second side surface
44c Third side surface
44d Base area
46 Connecting bracket
50 Blade holder
60 Threaded connection
EK Plane of the blade
EB Plane of the base element
lB Longitudinal axis of the base element
l1 First longitudinal axis
l2 Second longitudinal axis
α Angle

1. A surgical instrument comprising:
a handle;
a blade disposed thereon with a concavely developed cutting edge, wherein the blade comprises a blade plane,
wherein the surgical instrument comprises ultrasound visibility-enhancing media.

2. The surgical instrument as in claim 1, wherein the media comprise ultrasound reflectors.

3. The surgical instrument as in claim 2, wherein the ultrasound reflectors are developed as indentations.

4. The surgical instrument as in claim 3, wherein each indentation of the indentations comprise at least two side faces that are each oriented perpendicularly to one another.

5. The surgical instrument as in claim 1, wherein the media are disposed nondetachably on the blade.

6. The surgical instrument as in claim 1, wherein the media comprise, at least in sections, a coating.

7. The surgical instrument as in claim 1, wherein the media are at least in sections encapsulated with the blade.

8. The surgical instrument as in claim 1, wherein the media comprise a sheet-like base element comprising a base plane.

9. The surgical instrument as in claim 2, wherein the ultrasound reflectors are disposed on a base element.

10. The surgical instrument as in claim 8, wherein the base plane is substantially disposed perpendicularly to the blade plane.

11. The surgical instrument as in claim 3, wherein a first of the side faces of one of the indentations forms an angle of approximately 35° with the base plane.

12. The surgical instrument as in claim 3, wherein a second and a third of the side faces are disposed symmetrically to a longitudinal axis of the base element.

13. The surgical instrument as in claim 3, wherein the indentations comprise a triangular base area in an outer surface of the base element, wherein an apex of the triangle points to a distal end of the surgical instrument.

14. The surgical instrument as in claim 1, comprising a form of a stylus with a distal end and a proximal end, wherein the blade is disposed at the distal end.

15. The surgical instrument as in claim 1, wherein the blade is disposed on a blade holder.

16. The surgical instrument as in claim 15, wherein the media are nondetachably disposed on the blade holder.

17. The surgical instrument as in claim 1, wherein the blade or the blade holder is detachably secured on the handle.

18. The surgical instrument as in claim 15, comprising a first section, formed substantially by the handle with a first longitudinal axis and a second section, formed substantially by the blade holder, with a second longitudinal axis, wherein the second longitudinal axis is bent by an angle with respect to the first longitudinal axis, which angle is in the range of 10° to 40°.

19. The surgical instrument as claim 18, wherein the angle by which the second longitudinal axis is bent with respect to the first longitudinal axis lies in the blade plane.
